(19) United States
(12) Patent Application Publication
(10) Pub. No.: US 2006/0216795 A1
(43) Pub. Date: Sep. 28, 2006

(54) Sample preparation method, analysis system and preparation device

(75) Inventors: Katsuhiro Kanda, Hitachinaka (JP); Yukie Sasakura, Hitachinaka (JP); Tsuyoshi Ogino, Matsudo (JP); Makoto Nogami, Hitachinaka (JP)

Correspondence Address: MCDERMOTT WILL & EMERY LLP, 600 13TH STREET, N.W., WASHINGTON, DC, 20005-3096, US

(73) Assignee: HITACHI HIGH-TECHNOLOGIES CORPORATION

(21) Appl. No.: 11/356,114

(22) Filed: Feb. 17, 2006

(30) Foreign Application Priority Data
Mar. 25, 2005 (JP) 2005-088499

(51) Int. Cl. C12P 19/24

(52) U.S. Cl. 435/94

(57) ABSTRACT

The object of the present invention is to promote the efficiency and simplify a sample pretreatment step for analysis of molecules contained in the sample. In the present invention, a plurality of various enzymes necessary for pretreatment of sample for analysis of various molecules contained in the sample are immobilized on a solid phase separately for each kind of enzyme, and the enzyme reactions are carried out simultaneously. That is, a sample is reacted with plural kinds of enzymes under the same conditions, and contamination caused by the enzyme reactions is inhibited and the sample is subjected to a given pretreatment.

### INCORPORATION BY REFERENCE

The present application claims priority from Japanese application JP2005-088499 filed on Mar. 25, 2005, the content of which is hereby incorporated by reference into this application.

### BACKGROUND OF THE INVENTION

The present invention relates to a sample preparation method for analysis of various molecules contained in a sample, an analysis system, and a sample preparation device.

The mass spectrometry and liquid chromatography now become principal methods for analysis of various molecules in a sample. The mass spectrometry is a method of ionizing molecules to be analyzed and thereafter separating and detecting the molecules depending on the charge and mass number. On the other hand, the liquid chromatography is a method of capturing the molecules to be analyzed by a carrier which reacts with the molecules depending on the properties of the molecules and thereafter eluting the molecules under given conditions to separate and detect them.

In both of these methods, the sample must be subjected to pretreatment for analyzing the molecules in the sample. For example, when protein contained in the sample is analyzed, it is generally necessary to subject the protein to peptide fragmentation by a protease treatment or the like. Similarly, in the case of analyzing a sugar chain added to protein by post-translational modification, it is also necessary to previously isolate the sugar chain with glycopeptidase or the like and then recover the sugar chain (see JP-A-08-228795).

When molecules in a sample are analyzed by the above-mentioned separation and detection method, it is necessary to treat the molecules in the sample with various enzymes, and, in general, since optimum conditions of reaction differ for every enzyme, the reaction is usually carried out in liquid phase for every enzyme. As a result, although the enzyme reaction conditions can be adapted to the optimum conditions of each enzyme, steps such as preparation of enzyme solution and reaction increase to cause troublesomeness and, furthermore, the enzymes are abandoned in every operation. Moreover, since a step of recovering the reaction product for every reaction is added, decrease in quantity of recovered product is caused due to the decrease in recovery rate. In addition, the enzyme reaction carried out in liquid phase results in coexistence of enzymes in isolated state which are proteins other than the protein to be analyzed, giving adverse effect on accuracy of analysis at the step of separation and detection.

An object of the present invention is to solve the above problems and aim at high efficiency and simplification of the sample pretreatment step for analysis of molecules in the sample.

### SUMMARY OF THE INVENTION

The present invention for attaining the above object is characterized in that plural kinds of enzymes necessary for sample pretreatment for analysis of various molecules contained in the sample are immobilized separately for each kind of the enzyme on a solid phase, and enzyme reactions are carried out simultaneously. That is, it is an important characteristic of the present invention that a sample is subjected to a given pretreatment by reacting the sample with plural kinds of enzymes under the same conditions, thereby inhibiting contamination caused by the enzyme reactions.

According to the present invention, enhancement of efficiency and simplification of the pretreatment of sample can be attained.

Other objects, features and advantages of the invention will become apparent from the following description of the embodiments of the invention taken in conjunction with the accompanying drawings.

### BRIEF DESCRIPTION OF DRAWINGS

FIG. 1 shows oblique views which explain enzyme-immobilized supports used in the examples of the present invention, in which FIG. 1(a) shows an oblique view of enzyme-immobilized supports, each of which comprises one solid phase on which one kind of enzyme is independently immobilized, and FIG. 1(b) shows an oblique view of an enzyme-immobilized support comprising one solid phase on which plural kinds of enzymes are immobilized separately from each other.

FIG. 2 shows schematic sectional views of an enzyme reaction chamber, in which FIG. 2(b) is a side sectional view and FIG. 2(c) is a plan sectional view.

FIG. 3 is a schematic view of a reaction product analyzing system according to the example of the present invention.

FIG. 4 schematically shows a flow of preparation of enzyme-immobilized support according to the example.

FIG. 5 schematically shows a flow of an analytical operation using the enzyme-immobilized support of the experimental example.

FIG. 6 schematically shows an enzyme solution dispensing system according to the example.

FIGS. 7(a)-7(g) schematically show a flow of operational steps according to the example.

FIG. 8 is a graph showing the results of peptide analysis by HPLC according to the example.

FIG. 9 is a graph showing the results of analysis of ABOE-labeled sugar chain by HPLC according to the example.

### DESCRIPTION OF REFERENCE NUMERALS

1—Support (solid phase), 2—Enzyme A, 3—Enzyme B, 4—Enzyme X, 5—Enzyme-immobilized support, 6—Reaction chamber, 7—Reaction solution, 8—Reaction solution feed opening, 9—Reaction solution vibrating membrane, 10—Reaction solution vibrating device, 11—Temperature controlling device, 12—Enzyme reaction unit, 13—Sample, 14—Separation and detection unit, 15—Liquid chromatography, 16—Mass spectrometry, 17—System control device, 18—Control device, 19—The whole dispensing device, 20—Rail, 21—Position catching device, 22—Head, 23—Nozzle, 24—Nozzle washing device, 25—Nozzle drying device, 26—Various enzyme solution reservoir, 27—Pump, 28—Temperature/humidity sensor, 29—Seal for immobilization (hydrophobic sheet), 30—Well for immobilization of enzyme, 31—Various enzyme solutions, 32—Closed vessel, 33—Water drop for inhibition of drying, 34—Well for reaction, 35—Modified/reduced substrate solution, 36—Reaction product, 37—Peptide chromatogram, 38—Base line of absorption of ultraviolet region, 39—Isolated sugar chain chromatogram, 40—Base line at detection of fluorescent.

### DETAILED DESCRIPTION OF THE INVENTION

An example of the present invention will be explained in more detail using the drawings. The example should not be construed as limiting the invention.

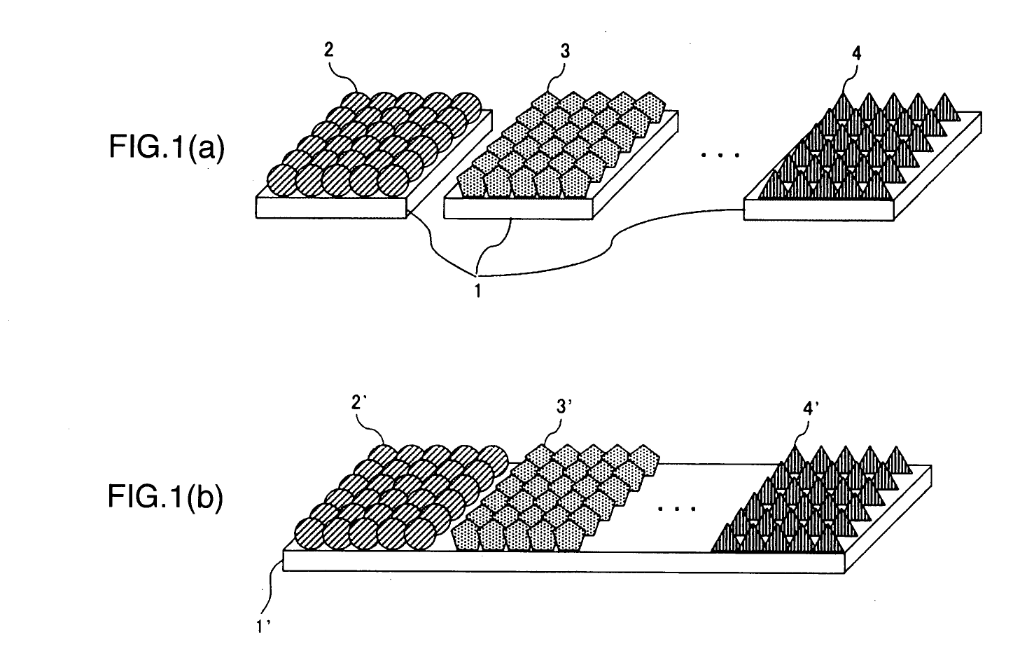

FIG. 1(a) shows the state of different kinds of enzymes 2, 3 and 4 being immobilized on solid phases 1, respectively, and the respective enzymes are selected depending on the molecules to be analyzed and the purpose. The kinds, amounts and dispositions of the enzymes are not limited. A plurality of these separate enzyme-immobilized supports are placed in combination in one reaction chamber and the enzymes are simultaneously contacted and reacted with a sample solution. Therefore, according to such a method, optional combination of the enzymes can be employed even if the kinds of the samples are different.

In FIG. 1(b), different kinds of enzymes 2′, 3′ and 4′ are separately immobilized on one support 1′. In this case, the sample can be simply subjected to pretreatment reaction using one enzyme-immobilized support.

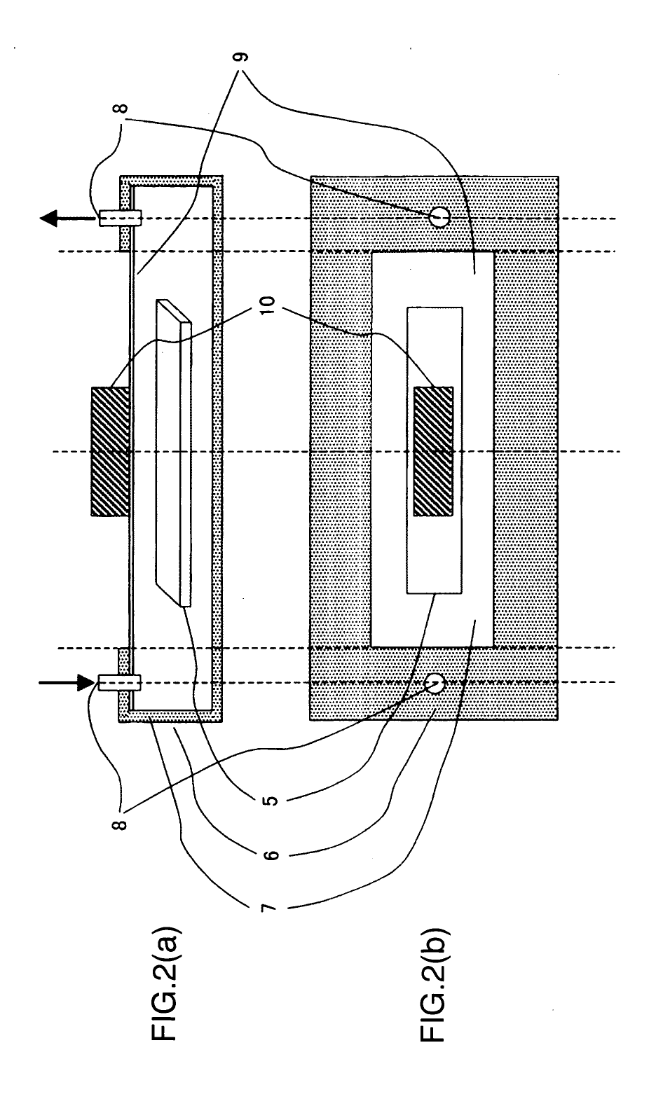

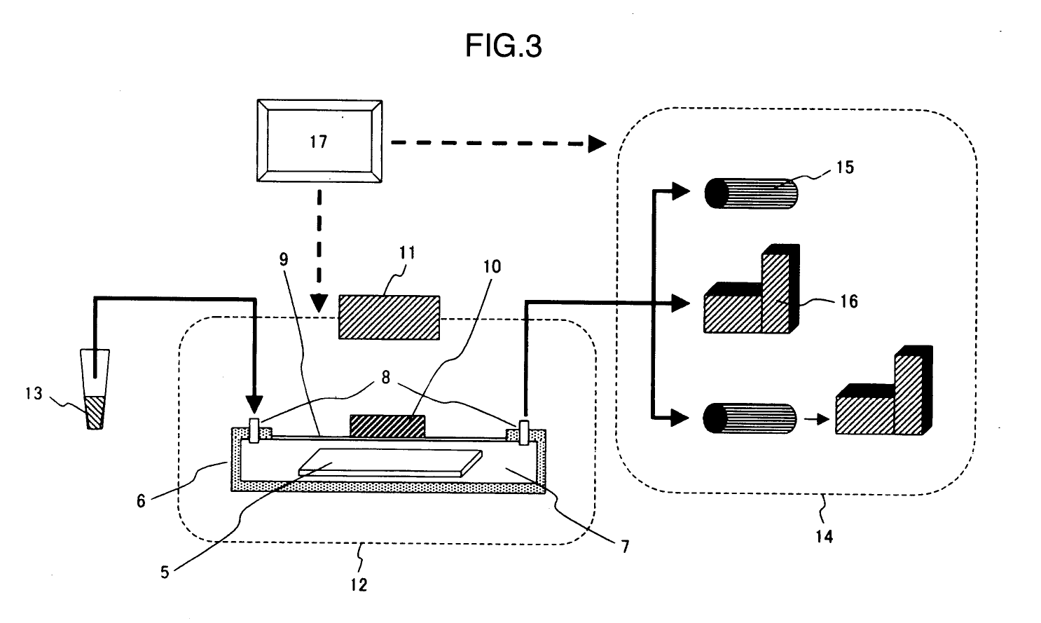

FIG. 2 shows sectional views of the enzyme reaction system of FIG. 3, in which FIG. 2(a) is a side sectional view and FIG. 2(b) is a plan sectional view. In FIG. 2 and FIG. 3, the same reference numerals indicate the same elements.

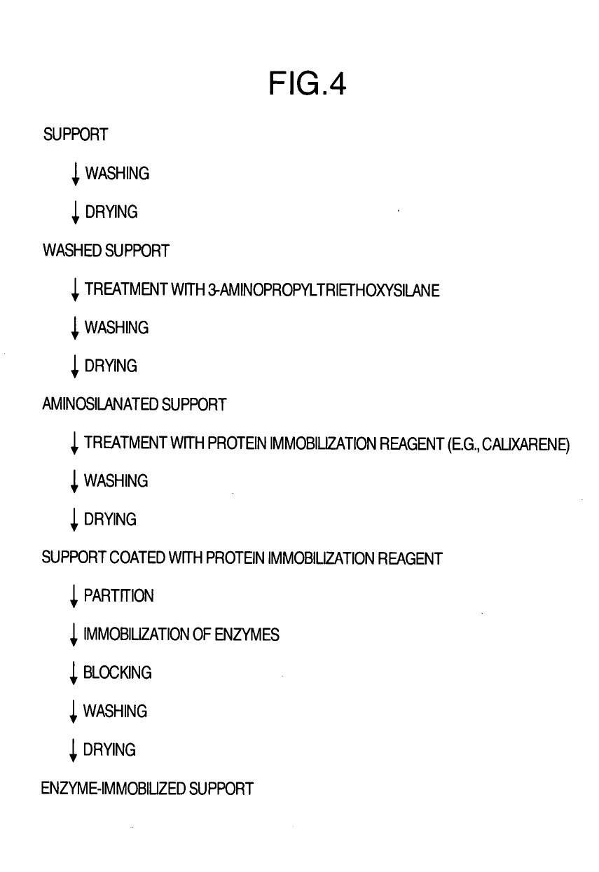

FIG. 4 shows an example of steps for production of enzyme-immobilized support. This production flow will be explained in the following example when occasion demands.

For example, proteases include trypsin, chymotrypsin, pronase, etc. and glyco isolating enzymes include glycopeptidase, etc. The present invention is not limited to these enzymes. For example, in the case of the sample being a glycoprotein, when peptide and sugar chain are to be recovered, two kinds of enzymes of trypsin and glycopeptidase can be combined and when amino acid and sugar chain are to be recovered, three kinds of enzymes of trypsin, pronase and glycopeptidase can be combined.

The solid phase 1 on which the enzymes 2, 3 and 4 are immobilized may comprise any materials as long as they can form a support. As the materials, mention may be made of, for example, glasses, resins, wafers and metals, and the shape of the support may be any of flat plate shape, spherical shape, channel shape, cylindrical shape, etc. The support may have any sizes as long as it can be used in the reaction chamber 6 and the like.

Immobilization of enzymes 2, 3 and 4 on the support 1 may be carried out by any methods in which the enzyme activity after immobilization can be maintained. For example, there are host-guest interaction method using calixarene, covalent bonding method using various functional groups such as aldehyde group and epoxy group, electrostatic bonding method using amino group and the like, adsorption method using nitrocellulose membrane, and others. An embedding method with polymer gel can also be utilized although enzymes may be partially covered to cause decrease of effective surface area.

For example, when enzymes 2, 3 and 4 are densely immobilized on the support 1 in the host-guest interaction method using calixarene, the blocking step (see FIG. 4) may be omitted in some cases.

In immobilization of enzymes 2, 3 and 4 on the support 1, there may occur functional inhibition due to degradation caused by the reaction of one enzyme with another enzyme depending on the kind of enzymes immobilized. Therefore, it is necessary to carry out the immobilization with separating the enzymes so that different kinds of enzymes do not contact with each other (see FIG. 1 and FIG. 4).

In FIG. 3, an enzyme-immobilized support 5 on which various kinds of enzymes are immobilized is disposed in a reaction chamber 6 and allowed to react with a reaction liquid 7. For some shapes of the supports 1 and 1′, such as channel shape and cylindrical shape, the supports 1 and 1′ per se may serve both as the enzyme-immobilized support and the reaction chamber 6.

There may be employed a method where plural kinds of the enzymes 2′, 3′ and 4′ are immobilized on one support 1′, and a plurality of enzyme reactions are carried out simultaneously using the resulting enzyme-immobilized support 5. Furthermore, there may be employed a method where one kind of the enzymes 2, 3 and 4 is immobilized on one support 1, respectively, and a plurality of enzyme reactions are carried out simultaneously by collecting the supports 1 on which the same kind of the enzyme is immobilized, respectively.

By simultaneously carrying out a plurality of the enzyme treatments to result in reduction of the number of the operation steps, shortening of the treating time and decrease of troubles can be attained, and, furthermore, there is no need to recover the reaction product for every enzyme reaction. As a result, loss in the recovery amount of the final reaction products caused by decrease of recovery rate can be reduced. Moreover, since the number of operations such as recovery and dispensation of the reaction products during operation can be reduced, contamination of the reaction products can be inhibited.

In addition, decrease of recovery amount of the final reaction products can be inhibited by carrying out simultaneously a plurality of the enzyme treatments, and thus the possibility of disappearing of the target molecules due to loss of the sample particularly when a trace amount of molecules are targeted can be removed. This is important, for example, for accelerating the search of biomarker or improving the accuracy in analysis such as profiling.

The temperature of the reaction solution 7 in the reaction chamber 6 is controlled to a temperature suitable for the reaction of the enzymes 2, 3 and 4, or 2′, 3′ and 4′ immobilized on the support 1 or 1′ by a temperature control device 11. The temperature condition may not necessarily be an optimum condition, and can be a condition under which a necessary and enough reaction is carried out. If necessary, there may be applied a method where the temperature is adapted to an optimum condition for the reaction by changing the set temperature during the enzyme reaction.

The reaction may be carried out under still conditions, but for improving the reaction efficiency, the reaction solution 7 is preferably agitated. Agitation of the reaction solution 7 is carried out, for example, by vibrating a reaction solution vibrating membrane 9 by a vibrating device 10 to feed and circulate the solution by vibration, pumping, or the like (see JP-A-2004-283083) or by agitation of the solution by shaking or rotation of the reaction chamber per se. After the agitation, the reaction is carried out for a given time to perform digestion of protein or isolation of sugar chain.

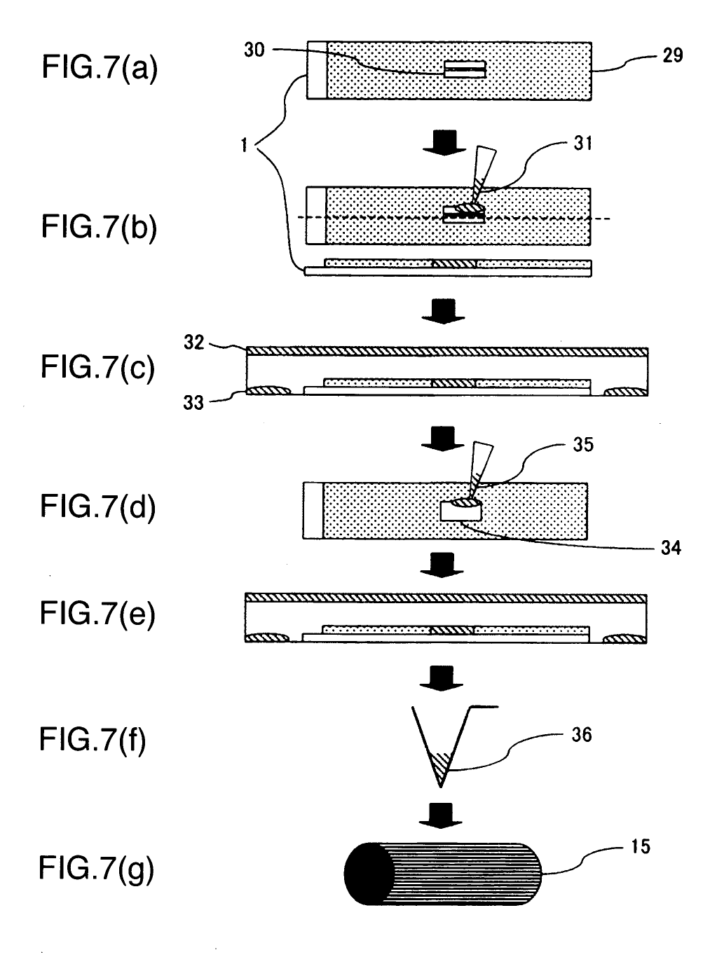

FIG. 7 is a flow chart of an experimental example, and (a) an enzyme immobilizing well 30 is formed in an enzyme immobilizing seal 29 (hydrophobic sheet) applied to the support 1, (b) an enzyme solution 31 is dropped in the enzyme immobilizing well 30, (c) then, the enzyme immobilized support is put in a closed vessel 32 containing water drop 33 for preventing the enzyme from drying, (d) a substrate solution 35 subjected to modification/reduction treatment is added to the immobilized enzyme in the reaction well 34, (e) in such a state, the enzyme and the sample are reacted, (f) a reaction product 36 is obtained, and (g) the reaction product 36 is recovered and used as a sample for separation and detection, for example, by mass spectrometry 16, liquid chromatography 15 or the like.

The reaction chamber 6 used in the present invention can be applied, for example, as a sample pretreatment unit 12 for mass spectrometry 16 or liquid chromatography 15. An enzyme reaction device 12 can be provided by automating or semiautomating a sample loading step of a sample 13, an enzyme reaction step including agitation of the reaction solution 7 fed to the reaction chamber 6 from the reaction solution feed opening 8 by the reaction solution vibrating device 10, and a reaction solution recovering step. Due to the enhancement of efficiency by using the enzyme-immobilized support 5, reduction in size of the apparatus can be realized, which makes it possible to handle the apparatus without securing a special installing place. On the other hand, it is also possible to optionally change the scale of the apparatus.

Furthermore, there can be provided a fully-automatic or semi-automatic reaction product analyzing system (FIG. 3) by automating the step of reloading of the recovered reaction product 36 to the various separation and detection devices 14. The separation and detection device 14 may be a liquid chromatography 15 or mass spectrometry 16 or a combination of them. The analyzing system in FIG. 3 is controlled by the system control device 17.

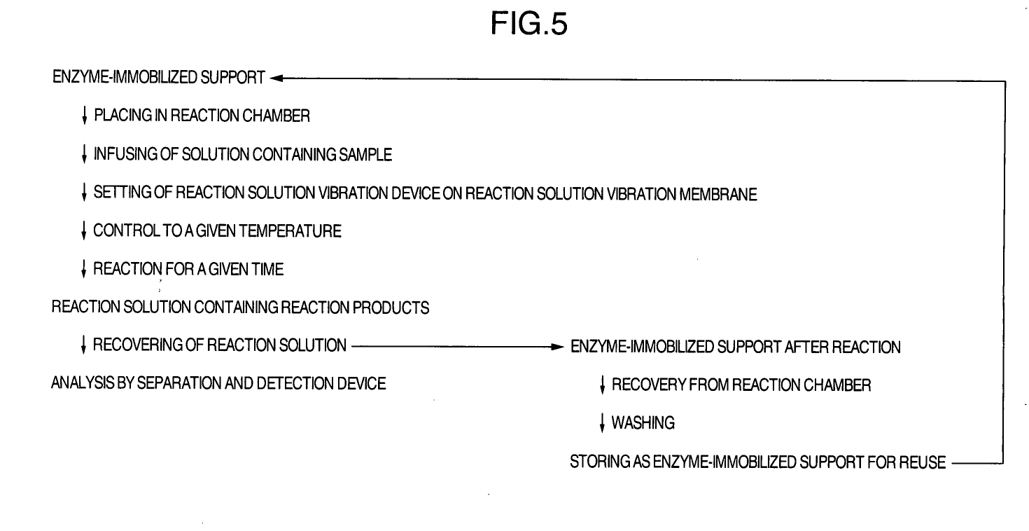

FIG. 5 is a flow chart explaining the pretreatment step of a sample according to the present invention. The enzyme-immobilized support 5 having various enzymes immobilized thereon in this example can be reused as long as the enzymes have activity (FIG. 5). Thus, the cost for analysis can be reduced. Moreover, as for the washing of the enzyme-immobilized support 5, the enzyme-immobilized support 5 can be easily washed by removing the support from the reaction chamber 6, and in the case of the enzyme-immobilized support 5 being integral with the reaction chamber 6, the support can be simply washed only by passing a washing solution. It should be noted that the reuse of the enzyme-immobilized support is not essential in this example.

By immobilizing various enzymes 2, 3 and 4, and 2′, 3′ and 4′ on the solid phases 1 and 1′ and using for reaction the enzyme-immobilized supports as in the present invention, incorporation of enzymes 2, 3 and 4, and 2′, 3′ and 4′ into the reaction product 36 can be avoided, and, hence, purification by removal of the enzymes is not needed and the results of separation and detection of the reaction product 36 are not affected by the incorporated enzymes.

The enzyme-immobilized support 5 having various enzymes immobilized thereon according to the present invention can provide a device suitable for the sample pretreatment unit 12 in which enzymes 2, 3 and 4, and 2′, 3′ and 4′ applicable to various analyses are immobilized in combination. Thus, the troubles to prepare the enzyme solution each time when it is required can be saved.

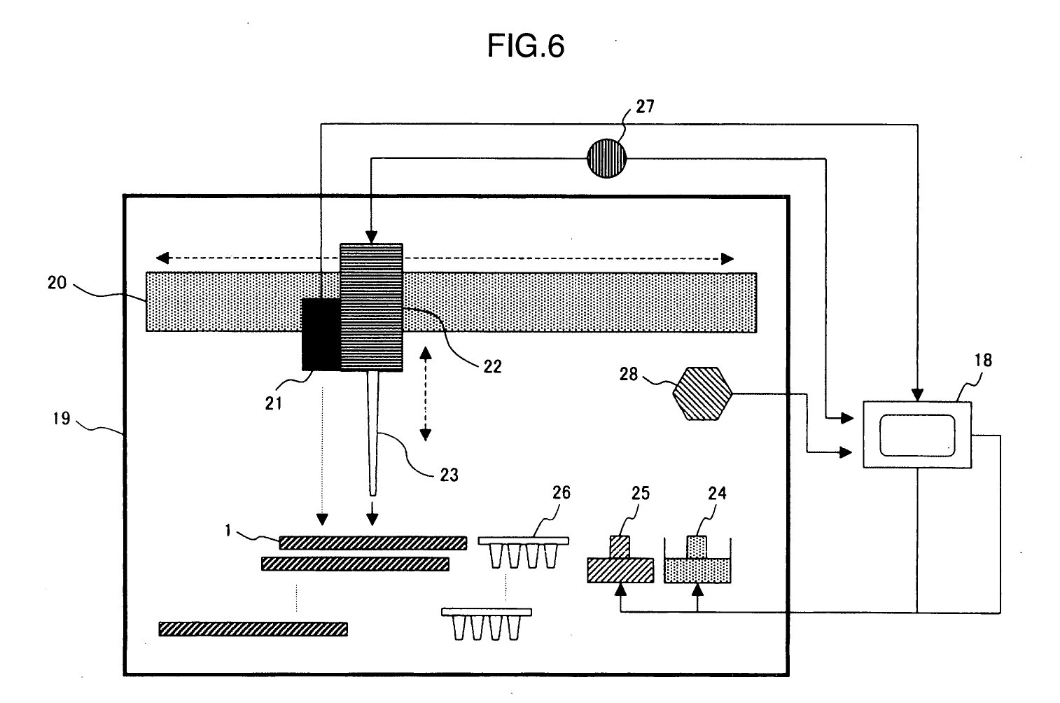

Furthermore, by using a solution dispensing device for preparing the enzyme-immobilized support 5 having various enzymes immobilized thereon (FIG. 6), an automatic or semiautomatic system for preparing the support can be constructed. In FIG. 6, a solution containing enzymes is supplied onto the support 1 from a head 22 having a nozzle 23 which is moved on a rail 20 by a position correcting device 21. The enzyme solution is dispensed by the nozzle 23 from an optionally selected enzyme solution reservoir 26. The nozzle 23 is washed by a nozzle washing device 24 and dried by a nozzle drying device 25 every time the enzyme solution is changed. The temperature and humidity in the dispensing device 19 are monitored by a temperature/humidity sensor 28. The pump 27 is connected with a necessary piping. The dispensing device is controlled by a controlling device 18.

According to the present invention, the pretreatment techniques for analysis of protein or sugar chain can be made higher in efficiency and simplified. Furthermore, recycling of the enzyme-immobilized support 5 having enzymes immobilized thereon can result in enhancement in efficiency and reduction in cost of analysis service business which is expected to advance into the fields of analysis of proteins or sugar chains in the future.

The present invention can be applied not only to analysis of proteins or sugar chains, but also to other fields which require an enzyme treatment step by changing the kind of the enzymes 2, 3 and 4, and 2′, 3′ and 4′ to be immobilized.

### EXPERIMENTAL EXAMPLE

In this experimental example, degradation to peptide and isolation of sugar chains were carried out using human immunoglobulin as a substrate by immobilizing, on the support 1 or 1′, trypsin which is an enzyme degrading the protein to peptide and glycopeptidase which is an enzyme isolating the sugar chains from protein or peptide.

[Immobilization of Enzyme on Support]

In this experiment, ProteoChip (Type A manufactured by Proteogen Inc.) was used as the support (1). Onto the ProteoChip was applied a immobilization seal 29 comprising a hydrophobic sheet 29 of 25 mm long×65 mm broad on which enzyme immobilizing wells 30 of 3 mm long×10 mm broad were disposed lengthwise in two rows at an interval of 1 mm. One of the enzyme immobilizing wells 30 was used for immobilization of trypsin and another was for immobilization of glycopeptidase.

Trypsin (T8802 manufactured by Sigma Co., Ltd.) was prepared at a concentration 1 mg/ml using a PBS buffer (pH 7.4) containing 50% glycerol, and glycopeptidase (1-365-193 manufactured by Roche Co., Ltd.) was prepared at a concentration of 0.67 U/μl using 100 mM sodium phosphate buffer (pH 7.2) containing 50% glycerol and 25 mM ethylenediamine-tetraacetic acid.

Each of the two kinds of these enzyme solutions 31 was separately added in an amount of 20 μl to each of the enzyme immobilizing wells 30 on the ProteoChip using a micropipetter. The ProteoChip was placed in a closed vessel 32 in which water drop 33 for inhibition of drying was previously provided at a corner, and incubation was carried out at 4° C. for one night to immobilize trypsin and glycopeptidase on the ProteoChip.

The ProteoChip was immersed in 50 ml of a washing solution [PBS buffer (pH 7.4)] and shaken for 5 minutes, and then the washing solution was exchanged and the ProteoChip was washed by shaking for further 5 minutes to remove unbonded enzymes. After completion of washing, the ProteoChip was rinsed twice with a reaction buffer [10 mM Tris-HCl buffer (pH 8.5)], followed by removing excessive water with a filter paper.

[Modification and Reductive Alkylation Treatment of Substrate Used for Enzyme Reaction]

A human immunoglobulin (14506 manufactured by Sigma Co., Ltd.) was used as a substrate. 1 mg of the substrate was weighed in a tube and diluted with 1 ml of a modification solution [0.2 mM Tris-HCl buffer (pH 8.5) containing 6M guanidine HCl and 2.5 mM ethylenediaminetetraacetic acid]. 1 μl of a reduction solution (sterilized water containing 60 mg/ml of dithiothreitol) was added thereto and nitrogen gas was gently blown upon the surface of the solution for 30 seconds. Then, the lid of the tube was covered with a Parafilm (manufactured by Pechiney Plastic Packaging Inc.), followed by allowing the tube to stand at 37° C. for 3 hours to subject the human immunoglobulin to modification and reduction treatments.

After the completion of the reaction, the treated human immunoglobulin was left to stand on ice for 5 minutes to lower the temperature of the solution, and then thereto was added 20 μl of an alkylation solution (a modification solution containing 50 mg/ml of iodoacetamide). Nitrogen gas was gently blown upon the surface of the solution for 30 seconds. Then, the lid of the tube was covered with a Parafilm, and the tube was left to stand at room temperature for 1 hour to alkylate the reduced amino acid residue. Guanidine HCl in the solution after the reaction was removed by repeating dialysis three times at 4° C. for 2 hours using 200 ml of a reaction buffer.

[Enzyme Treatment of Human Immunoglobulin with Immobilized Enzyme]

A part (1 mm long×10 mm broad) of hydrophobic sheet 29 divided as two enzyme immobilizing wells 30 for immobilization of trypsin and glycopeptidase was cut off by a blade to form a reaction well 34 (7 mm long×10 mm broad) in which a trypsin immobilized area and a glycopeptidase immobilized area were connected to each other.

50 μl of the human immunoglobulin 35 which had been subjected to reductive alkylation was added to the reaction well 34, and thereafter the ProteoChip was placed in a closed vessel 32 in which water drop 33 for inhibition of drying was previously dropped at a corner. Then, incubation was carried out at 37° C. for one night to react the substrate with the immobilized enzymes, and then the reaction product 36 was recovered.

[HPLC Analysis of Peptide Obtained by Digestion with Immobilized Trypsin]

365 μl of the reaction product was applied to a high-performance liquid chromatography (HPLC) 15 to inspect the presence of peptide which originated from the substrate and was obtained by digestion with the immobilized trypsin.

CAPCELLPAK C18 MG (2 mm×75 mm manufactured by Shiseido Co., Ltd.) was used as a column. Solution A (5% acetonitrile containing 0.1% TFA) and solution B (95% acetonitrile containing 0.1% TFA) were used as mobile phases. Linear gradient was carried out for 50 minutes from the beginning of elution in such a manner as the proportion of the solution A being from 100% to 60% (the proportion of the solution B being from 0% to 40%). Successively, peptide adsorbed to the column was eluted until lapse of 100 minutes after elution of 50 minutes from the starting of the elution under the condition of linear gradient in such a manner as the proportion of the solution A being from 60% to 0% (the proportion of the solution B being from 40% to 100%). The flow rate was 0.2 ml/min. Detection of the peptide was determined by absorbance for ultraviolet region (214 nm).

[ABOE Labeling of Isolated Sugar Chain Obtained by Treatment with Immobilized Glycopeptidase]

Labeling of isolated sugar chain was carried out for the total residual amount of the reaction product 36 excluding 5 μl used for the analysis of peptide. Using ABOE sugar chain labeling kit (manufactured by J-Oil Mills), the reducing terminal of isolated sugar chain was labeled with 4-aminobenzoic acid octyl ester (ABOE).

The reaction product 36 was transferred to a screw cap test tube and freeze-dried, followed by adding an ABOE reagent containing borane pyridine complex as a reducing agent and incubating at 80° C. for 30 minutes to carry out the reaction. After returning to room temperature, 1 ml of distilled water and 1 ml of chloroform were added, followed by agitating and then centrifuging to recover an aqueous layer. Furthermore, 1 ml of distilled water was added and then the same purification operation was repeated twice. The recovered aqueous layer was concentrated by freeze-drying and used for analysis using HPLC 15.

[HPLC Analysis of ABOE-Labeled Sugar Chain]

1/40 (in amount) of the resulting ABOE-labeled sugar chain was subjected to HPLC analysis to determine the presence of the isolated sugar chain which originated from the substrate and which was obtained by the immobilized glycopeptidase treatment.

HONEN PACK C18 (4.6 mm×75 mm, manufactured by J-Oil Mills) was used as a column. Solution A [a mixed solution of 0.1 M ammonium acetate buffer (pH 4.0):acetonitrile=75:25] and solution B [a mixed solution of 0.1 M ammonium acetate buffer (pH 4.0) acetonitrile=55:45] were used as mobile phases. Linear gradient was carried out for 10 minutes from the starting of elution in such a manner that the solutions were flowed at a constant concentration of the proportion of solution A being 90% (the proportion of the solution B being 10%), and, thereafter, the linear gradient was carried out until lapse of 50 minutes after elution of 10 minutes from the starting of the elution in such a manner as the proportion of the solution A being from 90% to 20% (the proportion of the solution B being from 10% to 80%), thereby eluting the ABOE-labeled sugar chain adsorbed to the column. The flow rate was 1 ml/min. The ABOE label was excited at 305 nm to detected a fluorescent of 360 nm.

[Results of Experiments]

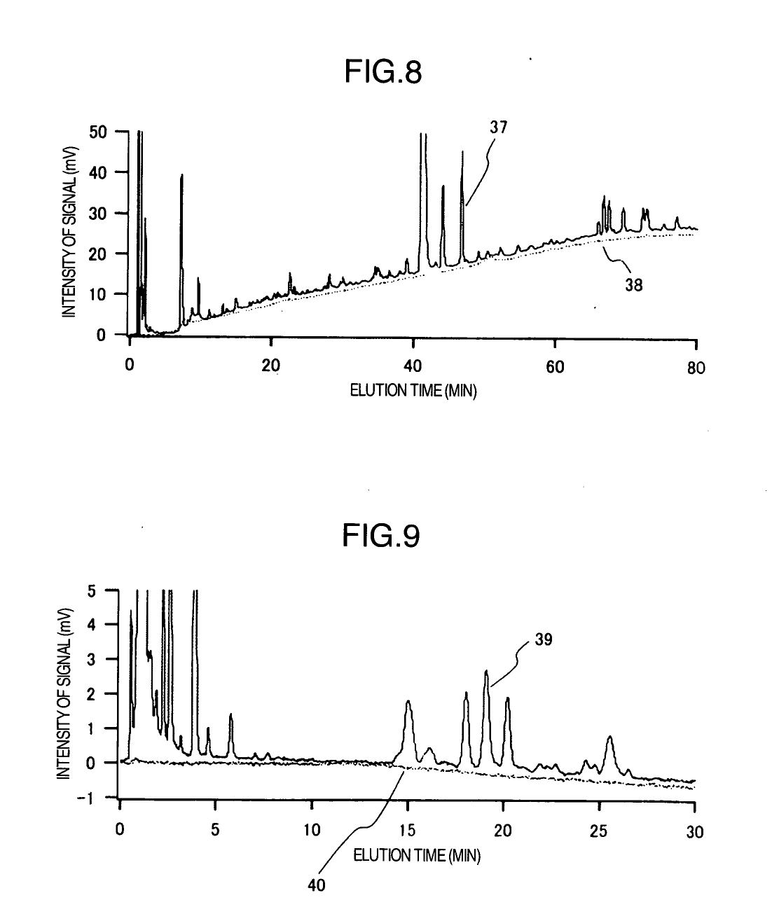

FIG. 8 shows peptide chromatogram 37 and base line 38 of absorption of ultraviolet region as the results of detection of peptide by HPLC 15. The respective fragments of the peptide obtained by digestion with immobilized trypsin were recognized between 5 minutes and 80 minutes of elution time, and thus this example shows that the human immunoglobulin which was a substrate was digested into peptide.

On the other hand, FIG. 9 shows isolated sugar chain chromatogram 39 and base line 40 in fluorescent detection as the results of detection of ABOE-labeled sugar chain by HPLC 15. The peaks of sugar chain which originated from the human immunoglobulin and which was isolated by treatment with the immobilized glycopeptidase were seen between 10 minutes and 30 minutes of the elution time, and thus this example shows that sugar chain can be isolated from the human immunoglobulin which was a substrate.

It was proved by the above experimental examples that the desired peptide and isolated sugar chain were obtained by separately immobilizing a plurality of enzymes (2′, 3′, 4′) on the support 1′ which were needed for digestion of protein and isolation of sugar chain and carrying out simultaneously both the enzyme reactions under the same conditions. This shows that this example can be applied to the pretreatment step of sample for the analysis of protein or sugar chain, and, furthermore, enhancement of efficiency and simplification of the step can be attained.

According to this example, plural kinds of enzymes necessary for pretreatment of sample for analysis of various molecules contained in the sample are immobilized on a solid phase separately for each kind of the enzyme, and the enzyme reactions are carried out simultaneously. Therefore, the promotion of efficiency and simplification of the sample pretreatment can be attained and, besides, reuse of enzymes becomes possible. Furthermore, decrease of analytical accuracy caused by coexistence of enzyme molecules in analysis can be inhibited.

The present invention can be utilized as pretreatment of analysis of samples, and, besides, can be widely applied to various fields such as basic researches, medical treatments, preparation of medicines, medical examination, and diagnosis. Particularly, the present invention can be utilized for pretreatment in analysis of biomolecules such as digestion of protein and isolation of modification molecules such as sugar chains, and relates to enzyme-immobilized supports, pretreatment system, and the like.

It should be further understood by those skilled in the art that although the foregoing description has been made on embodiments of the invention, the invention is not limited thereto and various changes and modifications may be made without departing from the spirit of the invention and the scope of the appended claims.

1. A method for pretreatment of a sample for analysis of the sample which comprises contacting a plurality of molecules to be analyzed which are contained in the sample with an enzyme-immobilized support comprising a solid phase having enzymes immobilized thereon, where the enzyme-immobilized support has two or more kinds of enzymes as a whole.

2. A method according to claim 1, wherein the enzyme-immobilized support comprises a solid phase on which two or more kinds of enzymes are immobilized separately for each kind of the enzyme.

3. A method according to claim 1, wherein the enzyme-immobilized support comprises one solid phase on which two or more kinds of enzymes are immobilized separately for each kind of the enzyme.

4. A method according to claim 1 which comprises simultaneously contacting the sample with two or more kinds of enzyme-immobilized supports, each of which comprises one solid phase having one kind of enzyme immobilized thereon.

5. A method according to claim 1, wherein a desired reaction product is obtained by simultaneously carrying out the reactions in a reaction chamber.

6. A method according to claim 1, wherein the enzymes are removed from the reaction product by simultaneously carrying out the reactions in a reaction chamber.

7. A method according to claim 1, wherein the enzyme-immobilized support is repeatedly used for the pretreatment.

8. A system for analysis of a sample in which an enzyme reaction device and a separation and detection device are provided, said enzyme reaction device includes a means for pretreatment and analysis of the sample by contacting a plurality of molecules to be analyzed which are contained in the sample with an enzyme-immobilized support comprising a solid phase having enzymes immobilized thereon, and said enzyme-immobilized support has two or more kinds of enzymes as a whole.

9. An analysis system according to claim 8, wherein the enzyme-immobilized support comprises a solid phase on which two or more kinds of enzymes are immobilized separately for each kind of the enzyme.

10. An analysis system according to claim 8, wherein the enzyme-immobilized support comprises one solid phase on which two or more kinds of enzymes are immobilized separately for each kind of the enzyme.

11. An analysis system according to claim 8 which comprises simultaneously contacting the sample with two or more kinds of enzyme-immobilized supports, each of which comprises one solid phase having one kind of enzyme immobilized thereon.

12. A device for pretreatment of a sample which comprises, as one set, two or more kinds of enzyme-immobilized supports, each of which has a solid phase and enzymes immobilized on the surface of the solid phase.

13. An enzyme-immobilized support for pretreatment of a sample which comprises one solid phase and two or more kinds of enzymes immobilized separately for each kind of the enzyme on the surface of the solid phase.
